SEQUENCE LISTING (36 sequences)

SEQ ID NO: 1 - RNA, 21 nt, Artificial Sequence
  hsa-miR-552
aacaggugacugguuagacaa

SEQ ID NO: 2 - RNA, 24 nt, Artificial Sequence
  hsa-miR-1254
agccuggaagcuggagccugcagu

SEQ ID NO: 3 - RNA, 22 nt, Artificial Sequence
  hsa-miR-378
cuccugacuccagguccugugu

SEQ ID NO: 4 - RNA, 22 nt, Artificial Sequence
  hsa-miR-548n
caaaaguaauuguggauuuugu

SEQ ID NO: 5 - RNA, 17 nt, Artificial Sequence
  hsa-miR-1281
ucgccuccuccucuccc

SEQ ID NO: 6 - RNA, 22 nt, Artificial Sequence
  hsa-miR-26a
uucaaguaauccaggauaggcu

SEQ ID NO: 7 - RNA, 21 nt, Artificial Sequence
  hsa-miR-26b
uucaaguaauucaggauaggu

SEQ ID NO: 8 - RNA, 22 nt, Artificial Sequence
  hsa-miR-29a
acugauuucuuuugguguucag

SEQ ID NO: 9 - RNA, 22 nt, Artificial Sequence
  hsa-miR-30c-1
cugggagaggguuguuuacucc

SEQ ID NO: 10 - RNA, 22 nt, Artificial Sequence
  hsa-miR-30c-2
cugggagaaggcuguuuacucu

SEQ ID NO: 11 - RNA, 22 nt, Artificial Sequence
  hsa-miR-96
aaucaugugcagugccaauaug

SEQ ID NO: 12 - RNA, 23 nt, Artificial Sequence
  hsa-miR-105
ucaaaugcucagacuccuguggu

SEQ ID NO: 13 - RNA, 22 nt, Artificial Sequence
  hsa-miR-146a
ugagaacugaauuccauggguu

SEQ ID NO: 14 - RNA, 22 nt, Artificial Sequence
  hsa-miR-200b
uaauacugccugguaaugauga

SEQ ID NO: 15 - RNA, 23 nt, Artificial Sequence
  hsa-miR-200c
uaauacugccggguaaugaugga

SEQ ID NO: 16 - RNA, 22 nt, Artificial Sequence
  hsa-miR-223
ugucaguuugucaaauacccca

SEQ ID NO: 17 - RNA, 20 nt, Artificial Sequence
  hsa-miR-371-5p
acucaaacugugggggcacu

SEQ ID NO: 18 - RNA, 22 nt, Artificial Sequence
  hsa-miR-570
cgaaaacagcaauuaccuuugc

SEQ ID NO: 19 - RNA, 22 nt, Artificial Sequence
  hsa-miR-580
uugagaaugaugaaucauuagg

SEQ ID NO: 20 - RNA, 21 nt, Artificial Sequence
  hsa-miR-607
guucaaauccagaucuauaac

SEQ ID NO: 21 - RNA, 22 nt, Artificial Sequence
  hsa-miR-429
uaauacugucugguaaaaccgu

SEQ ID NO: 22 - RNA, 22 nt, Artificial Sequence
  hsa-miR-452
cucaucugcaaagaaguaagug

SEQ ID NO: 23 - RNA, 21 nt, Artificial Sequence
  hsa-miR-488
uugaaaggcuauuucuugguc

SEQ ID NO: 24 - RNA, 20 nt, Artificial Sequence
  hsa-miR-518a-5p
cugcaaagggaagcccuuuc

SEQ ID NO: 25 - RNA, 20 nt, Artificial Sequence
  hsa-miR-527
cugcaaagggaagcccuuuc

SEQ ID NO: 26 - RNA, 22 nt, Artificial Sequence
  hsa-miR-539
ggagaaauuauccuuggugugu

SEQ ID NO: 27 - RNA, 22 nt, Artificial Sequence
  hsa-miR-548a-5p
aaaaguaauugcgaguuuuacc

SEQ ID NO: 28 - RNA, 22 nt, Artificial Sequence
  hsa-miR-548b-5p
aaaaguaauugugguuuuggcc

SEQ ID NO: 29 - RNA, 22 nt, Artificial Sequence
  hsa-miR-548d-5p
aaaaguaauugugguuuuugcc

SEQ ID NO: 30 - RNA, 21 nt, Artificial Sequence
  hsa-miR-553
aaaacggugagauuuuguuuu

SEQ ID NO: 31 - RNA, 21 nt, Artificial Sequence
  hsa-miR-559
uaaaguaaauaugcaccaaaa

SEQ ID NO: 32 - RNA, 20 nt, Artificial Sequence
  hsa-miR-609
aggguguuucucucaucucu

SEQ ID NO: 33 - RNA, 21 nt, Artificial Sequence
  hsa-miR-629
uggguuuacguugggagaacu

SEQ ID NO: 34 - RNA, 22 nt, Artificial Sequence
  hsa-miR-643
acuuguaugcuagcucagguag

SEQ ID NO: 35 - DNA, 151 bp, Artificial Sequence
  CYP2E1 3-untranslated region
gtgtgtggaggacaccctgaaccccccgctttcaaacaagttttcaaattgtttgaggtcaggatttctcaaactgattcctttctttgcatatgagtatttgaaaataaatatttcccagaatataaataaatcatcacatgattatttt

SEQ ID NO: 36 - DNA, 1000 bp, Artificial Sequence
  CYP2E1 5-flanking region
agaacttctatatattgccagttagaagacagaatgaaaaacattctcttcattctaaccacacacacaaaaaagctccacaaaatacctatggactaccttcatagaaggtggaagagggtctgtatgaagaaaataattaatacatgaaagaagaagctagtcaatgtggagctctattgtgtcccgggatcaacaaagacaagatatctttaaaatcgtcttctaaatttaccctaatgtaaaacaaatccaataaaactctaatgtaattttttaagaatttaaatttggaataattccaaagaacaatttttcttaattttctacagccagaatatatacctttaaaaaaaatgaaaacagagattaactttctcagaattggttgactcactctttccttttatttttcttccatggaattttccagttaacttgagaaagtggaatcgaattccgatgttgaattttccttctggccccattcatgtggcaggtggtgattcaggtactactgggggctgctcagacaaacctcctcatcagacatcaagaggctgttgcaccaggagggccggtaccgtgtctagaggtggtcggcatggggttggagttgtattacataaaccctactccaaacaaatgcatggggatgtggctggagttccccgttgtctaaccagtgccaaagggcaggacggtacctcaccccacgttcttaactatgggttggcaacatgttcctggatgtgtttgctggcacagtgacaggtgctagcaaccagggtgttgacacagtccaactccatcctcaccaggtcactggctggaacccctgggggccaccattgcgggaatcagcctttgaaacgatggccaacagcagctaataataaaccagtaatttgggatagacgagtagcaagagggcattggttggtgggtcaccctccttctcagaacacattataaaaaccttccgtttccacaggattgt

(19) United States
(12) Patent Application Publication
(10) Pub. No.: US 2015/0252365 A1
(43) Pub. Date: Sep. 10, 2015

(54) METHOD FOR SCREENING MICRORNAS WITH GENE SILENCING FUNCTION AT BOTH LEVELS OF TRANSCRIPTION AND POST-TRANSCRIPTION

(71) Applicant: SHANGHAI INSTITUTE OF MATERIA MEDICA, CHINESE ACADEMY OF SCIENCES, Pudong, Shanghai (CN)

(72) Inventors: Jin Ren, Pudong (CN); Yizheng Wang, Pudong (CN); Lingling Miao, Pudong (CN); Likun Gong, Pudong (CN); Xinming Qi, Pudong (CN); Chenggang Li, Pudong (CN); Guozhen Xing, Pudong (CN)

(21) Appl. No.: 14/416,559

(22) PCT Filed: Jul. 26, 2013
(86) PCT No.: PCT/CN2013/080160

§ 371 Date: May 14, 2015

(30) Foreign Application Priority Data
Jul. 26, 2012 (CN) 201210262337.0

(51) Int. Cl. C12N 15/113

(52) U.S. Cl. CPC C12N 15/113; C12N 2320/11; C12N 2310/141

(57) ABSTRACT

Provided is a method for screening microRNAs with gene silencing function at both transcriptional and post-transcriptional levels. The microRNA obtained via the screening method can down-regulate the expression of mRNA of a target gene by binding to a specific site of 5′-flanking region of the target gene, and also down-regulate the expression of the protein of the target gene by binding to a specific site of the 3′-untranslated region of the target gene. Also provided are microRNAs obtained via the screening method, the use of the same in gene silencing, and a method of gene silencing by using the same.

This application is a national stage application of International Patent Application No. PCT/CN2013/080160, filed Jul. 26, 2013, which claims priority to Chinese Patent Application No. 201210262337.0, filed Jul. 26, 2012. The entirety of the aforementioned applications is incorporated herein by reference.

### FIELD OF INVENTION

This invention belongs to the field of biotechnology, and specifically, relates to a method for screening microRNAs with gene silencing function at both transcriptional and post-transcriptional levels. The obtained microRNAs can regulate the expression of the target gene at both transcriptional and post-transcriptional levels (dual regulation).

### BACKGROUND ART

Gene silencing is a generalized term to describe the epigenetic regulation of gene expression. In general, this term refers to the phenomenon that a certain gene in an organism is not expressed or the expression thereof is lower than the normal level, which is not due to the alteration of the gene itself Gene silencing, as an endogenous mechanism of gene expression and regulation, widely exists in organisms, and is also one of important techniques for suppressing the gene expression in biological research. It is a very effective way to study the function of a gene by decreasing the expression level of the gene and then observe the phenomenon therefrom. Various drugs for silencing a certain gene, have been developed by targeting nucleic acid. Currently, several types of oligonucleotides, such as antisense oligonucleotides, small interfering RNAs (siRNAs), aptamers, spiegelmer microRNAs and the like, have been implicated in clinical trials.

Gene silencing may be realized in various manners, and can occur during either transcription or post-transcription. Transcriptional gene silencing includes histone modification, genetic imprinting, paramutation, transposon silencing, transgene silencing, position effect, RNA mediated DNA methylation, etc. Post-transcriptional gene silencing is mainly achieved by RNA interference, nonsense strand mediated RNA decay or the like. Up to now, RNA interference (RNAi) is the most commonly used post-transcriptional gene silencing technique. Introduction of foreign small fragment double-stranded RNAs (dsRNAs) or small hairpin RNAs (shRNAs) homologous to endogenous mRNAs into cells may induce the degradation of the mRNAs, so as to block the expression of the corresponding gene. Finally, both dsRNAs and shRNAs may be processed into functional double-stranded small interfering RNAs (siRNAs) having the length of 21 by Dicer.

SiRNA-mediated gene silencing technique, which has been widely used in scientific study of gene function so far, is the most commonly used. Meanwhile, siRNAs are of important significance in therapeutics and drug development for various diseases, such as viral infection, tumors and the like. However, RNAi technique has its limitations in that (1) RNAi cannot completely block expression of a gene, in particular, a high-expressed genes, in mammalian cells; (2) RNAi is not suitable for all genes, for example, dsRNAs does not have a significant inhibitory action on some genes functioning in neuron cells; (3) Off-target effects are significant in RNAi; (4) SiRNAs may activate genes for in vivo interferon response to induce side effects.

MicroRNA (miRNA) is a big family of endogenous small non-coding RNA recently found regulating gene expression, which functions typically in suppressing gene expression. It has been found so far that miRNAs regulate the expression of about 60% or more of the whole genome, and thus are involved in almost all the living processes of cells including cellular proliferation, differentiation, apoptosis, metabolism and the like, and also play an important role in pathological processes of cardiovascular diseases, nervous system diseases, hematopoietic system diseases, diabetes, and various tumors. As endogenous small RNAs have good suitability and specificity and significant inhibitory effects, the development of miRNAs as a drug for gene silencing is of great interest. So far, there are several microRNAs and modified sequences thereof as drugs under investigation in several clinical trials.

Function Mechanism of miRNAs

The commonly known gene silencing mechanism of miRNAs is the post-transcriptional gene silencing. The target of miRNAs is mostly located in the 3′-untranslated region (3′UTR) of mRNAs. A functional single-strand mature miRNA is integrated into RNA-induced silencing complex (RISC) and binds the target mRNAs, and then suppresses post-transcriptional gene expression through mRNA shearing, mRNA deadenylation, translation inhibition and the like. Such regulation of mRNA depends on the complementary pairing of the ‘seed region’ sequence (nucleotide 2-7) of the miRNA and the 3′-untranslated region sequence of the target mRNA.

Recently, it was reported that miRNA can also target gene promoter region via complementary pairing, results in the emichment of histone methyltransferases or DNA methyltransferases (DNMTs) in the region, affects the binding of RNA polymerases and causes histone or DNA methylation, and thereby suppresses gene transcription at transcriptional level. In some reports, such kind of regulation is independent of the sequence pairing of miRNA seed region and can suppress the gene expression at a lower dose in a long time. Therefore, miRNA can silence target gene by transcriptional or post-transcriptional suppression.

### SUMMARY OF INVENTION

The present inventors carried out a large amount of experiments and identifications, and screened out miRNAs that can action on one target gene at two levels, i.e., both transcriptional suppression and post-transcriptional suppression. This foreign microRNA can effectively inhibit target gene expression by suppressing both transcription and translation of the target gene, suggesting that the regulatory manner of microRNA on target gene at both transcriptional and post-transcriptional levels be an effective means for gene silencing.

Accordingly, one object of this invention is to provide a method for screening microRNAs with gene silencing function at both transcriptional and post-transcriptional levels.

Another object of this invention is to provide microRNAs screened out by the above-mentioned method.

Still another object of this invention is to provide the use of the microRNAs screened out by the above-mentioned method.

Still another object of this invention is to provide a method of gene silencing.

In one aspect of this invention, provided is a method for screening out a microRNA with gene silencing function at both transcriptional and post-transcriptional levels, comprising:

Step 1): predicting the microRNA binding site on the 5′-flanking region (5′-FR), on which a promoter may be probably located, of 1 to 2 kb upstream from the transcription start site of target gene, by using miRbase database, to identify the microRNA capable of binding to the promoter region of the target gene;

Step 2): predicting the microRNA binding site on the 3′-untranslated region (3′UTR) of the target gene by using Targetscan database to identify the microRNA capable of binding to the 3′-untranslated region of the target gene;

Step 3): chemically synthesizing the microRNA identified in Steps 1) and 2), evaluating its effects on the protein expression level of the target gene, and thereby screening out the microRNA capable of down-regulating the protein expression of the target gene;

Step 4): evaluating the effects of the microRNA capable of down-regulating the protein expression level of the target gene and obtained from Step 3) on the mRNA expression level of the target gene, and thereby screening out the microRNA capable of down-regulating both mRNA and protein levels of the target gene;

Step 5): evaluating the microRNA for its ability to silence the target gene at both transcriptional and post-transcriptional levels by using site-directed mutagenesis and luciferase reporter gene methods, and thereby screening out the microRNA with gene silencing function at both transcriptional and post-transcriptional levels.

In Step 1) of the method of the present invention, the miRbase database can be found at http://www.mirbase.org/search.shtml.

In Step 2) of the method of the present invention, the Targetscan database can be found at http://www.targetscan.org/.

The above-mentioned databases are most widely used and mostly recognized in the field of scientific research and drug development, and thus are adopted in the present invention for precise and reliable miRNA prediction.

In Step 3) of the method of the present invention, the method for evaluating the effects of microRNA on the protein expression level of the target gene includes liposome transfection, protein immunoblot and the like.

In Step 4) of the method of the present invention, the method for evaluating the effects of the microRNA on the mRNA expression level of the target gene includes total RNA isolation, cDNA reverse transcription and real-time fluorescence quantitative PCR.

In Step 5) of the method of the present invention, further included are the following steps:

(a) identifying the screened binding site of the 5′-flanking region of the target gene with the screened microRNA by using miRbase database prediction in combination with artificial alignment, mutating the binding site in the 5′-flanking region by using site-directed mutagenesis, inserting wild-type or mutated 5′-flanking regions into the renilla luciferase-carrying vector (PGL4.10, Promega) respectively, with the firefly luciferase-carrying vector PGL4.73 used as an internal reference, transforming the two types of vectors, PGL4.10 and PGL4.73, into cells respectively, introducing the microRNA obtained from Step 4), evaluating the effect of the mutation of the microRNA binding site in the 5′ flanking region on the renilla luciferase activity, confirming the binding site of the 5′ flanking region of the target gene with the microRNA, and finally determining whether the microRNA has the regulatory ability at transcriptional level, and thereby screening out microRNA that has the regulatory ability at transcriptional level.

(b) identifying the binding site of the 3′UTR of the target gene with the screened microRNA using Targetscan database prediction in combination with artificial alignment, mutating the binding site in the 3′UTR via site-directed mutagenesis, inserting the wild-type or mutated 3′UTR into the firefly luciferase-carrying vector (psiCHECK2) respectively, with the renilla luciferase self-carried by the vector used as an internal reference, transfecting the psiCHECK2 vectors carrying the wild-type or mutated 3′UTR into cells with the microRNA mimics, evaluating the effect of the mutation of the 3′UTR of the target gene on miRNA-induced inhibition on firefly luciferase activity, confirming the binding site of the microRNA having the regulatory ability at transcriptional level obtained from step (a) to the 3′UTR of the target gene, and finally determining whether the microRNA has regulatory ability at post-transcriptional (translational) level, and thereby screening out the microRNA with gene silencing function at both transcriptional and post-transcriptional levels.

As used herein, the term “artificial alignment” refers to examining the binding ability and pairing sequence of a microRNA to a target gene by using Vector NTI software (Invitrogen), RNA hybrid on-line software (http://bibiserv.techfak.uni-bielefeld.de/rnahybrid/) and handwork search for nucleotide sequence fragments (Excel, Microsoft), based on the above-mentioned databases (miRbase, TargetScan).

In the present invention, the mentioned cell may be PLC/PRF/5 liver cell line, 293 cell line, or 786-O renal cell line.

Therefore, the microRNAs screened out and obtained via the above method can not only down-regulate the mRNA expression of the target gene by binding to a specific site of 5′-flanking region of the target gene, that is, has inhibitory ability at transcriptional level, but also down-regulate the protein expression of the target gene by binding to a specific site of the 3′-untranslated region of the target gene, that is, has inhibitory ability at post-transcriptional/translational level.

In Step 4), a microRNA capable of down-regulating both mRNA and protein levels is obtained. The decline in mRNA level may be relevant to the regulatory mechanism of microRNA at transcriptional level (i.e. binding to the promoter or 5′-flanking region), and the decline in protein level may correlate with the regulatory mechanism of microRNA at post-transcriptional level. Therefore, the action mechanism of microRNA is testified using site-directed mutagenesis and luciferase reporter gene methods, based on the following reasons.

I. There are some limitations in determining the binding site between a microRNA and a target gene by using miRbase and TargetScan database prediction. For example, TargetScan database generally selects the predicted results by using the base pairing number, which is generally demanded as more than 6, of microRNA seed region and 3′-untranslated region. However, microRNA might be effectively bound to the 3′-untranslated region of the target gene to down-regulate the expression of the target gene even that the base pairing number is less than 6 or when U-G pairing exists, because the secondary structure of RNA is complex. In this case, the data may be better processed using artificial alignment to some extent.

II. The predicted binding site of the screened microRNA to the 5′-flanking region of the target gene is identified by using miRbase database prediction in combination with artificial alignment. The binding site in the 5′-flanking region is mutated by using site-directed mutagenesis. Then the 5′-flanking regions of wild-type and mutated 5′-flanking region are respectively inserted into luciferase-carrying vectors, and the vectors are transformed into cells. MicroRNA mimics are introduced and the effects of the mutation of the binding site of the 5′ flanking region on the luciferase activity are evaluated. The binding site of the microRNA to the 5′ flanking region of the target gene are identified, and finally whether the microRNA has regulatory ability at transcriptional level is determined.

III. The predicted binding site of the screened microRNA to the 3′UTR of the target gene is identified by using Targetscan database prediction in combination with artificial alignment. The binding site in the 3′UTR is mutated by using site-directed mutagenesis. Then the wild-type or mutated 3′UTR are respectively inserted into the luciferase-carrying vectors, and the vectors are transformed into cells. MicroRNA mimics are introduced and the effects of the mutation of the microRNA binding site of the 3′UTR on the luciferase activity are evaluated. The binding site of the microRNA to the 3′UTR of the target gene are identified, and finally whether the microRNA has regulatory ability at post-transcriptional (translational) level can be determined.

Based on the experimental results of I-III, a microRNA capable of regulating the expression of target gene at both transcriptional and post-transcriptional (translational) levels is obtained.

In this invention, the target gene can be any gene without limitation. Preferably, the target gene is cytochrome P450 2E1 (CYP2E1) gene or haemoxygenase 1(HMOX1) gene.

In another aspect of this invention, provided is a miRNA obtained by the above-mentioned method.

In this invention, preferably, CYP2E 1 gene is used as the target gene to screen out microRNA-552 (miR-552) having gene silencing function at both transcriptional and post-transcriptional levels. Or, for example, HMOX1 gene is used as the target gene to screen out microRNA-1254 (miR-1254) having gene silencing function at both transcriptional and post-transcriptional levels.

In another aspect of this invention, provided is the use of the miRNA obtained by the above-mentioned method for gene silencing. Accordingly, the microRNAs of this invention may be used in the development and screening of drugs for treating various diseases.

In a further aspect of this invention, provided is a method for gene silencing comprising performing gene silencing by using the miRNA obtained by the above-mentioned method.

Compared to siRNA, miRNA is an endogenous small RNA molecule with good suitability and specificity and significant inhibitory effects. The miRNA obtained by applying the method of this invention has better effects in gene silencing, compared to the previously reported miRNA for the same target gene (FIG. 9). This invention provides a new and effective method for screening microRNA capable of giving better silencing effects. This invention provides a new design for gene silencing commonly used in scientific research and for the development of microRNA drug for diseases.

### DESCRIPTION OF DRAWINGS

Hereinafter, a detailed description will be given in conjunction with the examples and drawings, which, however, are only for illustrative purposes and are not intended to limit the scope of the invention.

FIG. 1 is a flow chart schematically showing the method for screening a microRNA with gene silencing function at both transcriptional and post-transcriptional levels of the present invention.

FIG. 2 is a schematic diagram showing the microRNA obtained by predicting in 5′-flanking region (promoter region) of CYP2E1 by using miRbase database, and the binding site in the 5′-flanking region, wherein 5′FR represents 5′-flanking region.

FIG. 3 is a schematic diagram showing the microRNA obtained by predicting in 3′-untranslated region of CYP2E1 by using Targetscan database, and the binding site in 3′-untranslated region, wherein 3′UTR represents 3′-untranslated region.

FIG. 4 shows the effect of the microRNA obtained via prediction on protein expression of CYP2E1, wherein:

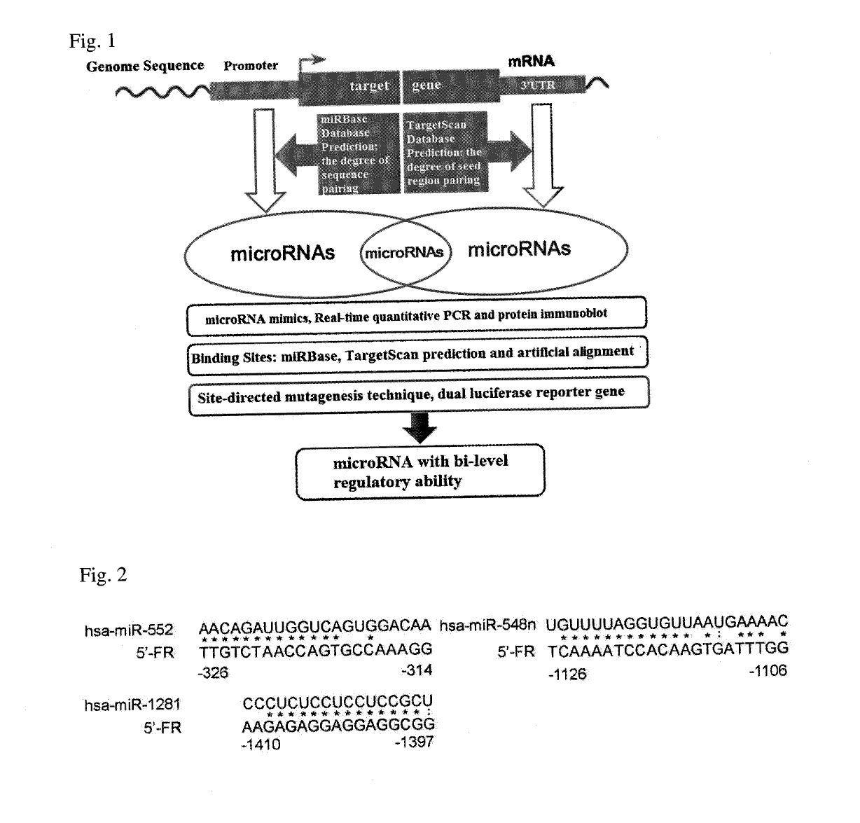

A. Protein immunoblot of evaluating the expression of CYP2E1 after transfecting each of miRNAs into PLC/PRF/5 cells, wherein GAPDH was used as the control of loading amount, the numbers in the figure refer to the names of the microRNAs, and nc is negative control.

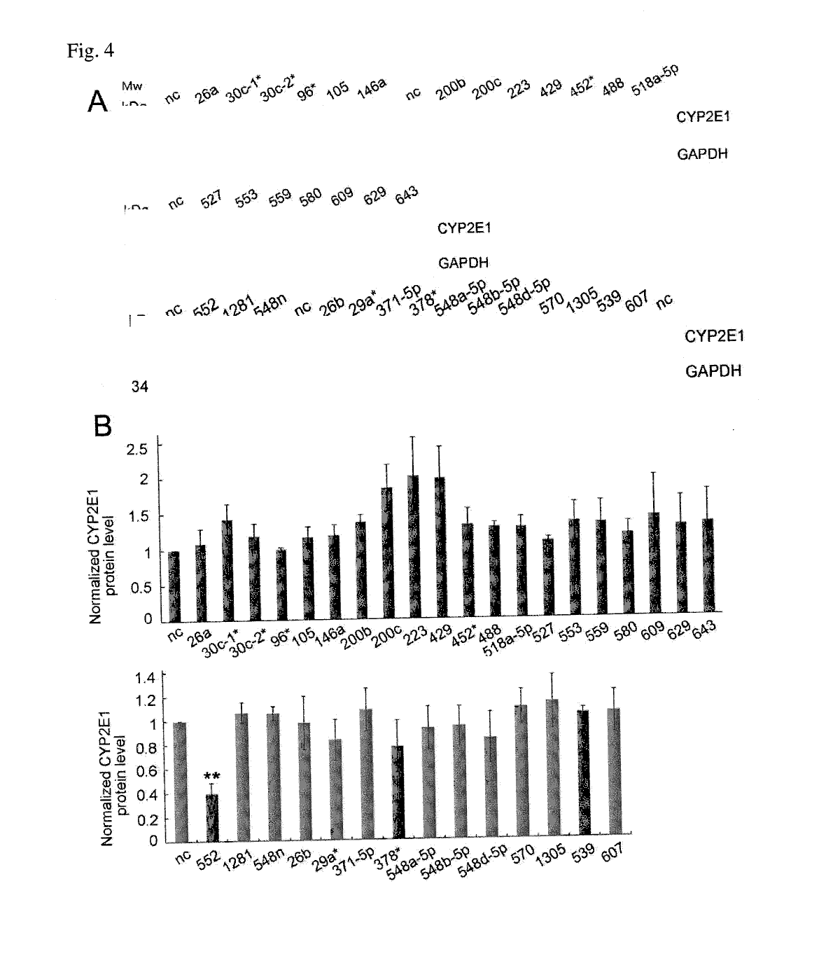

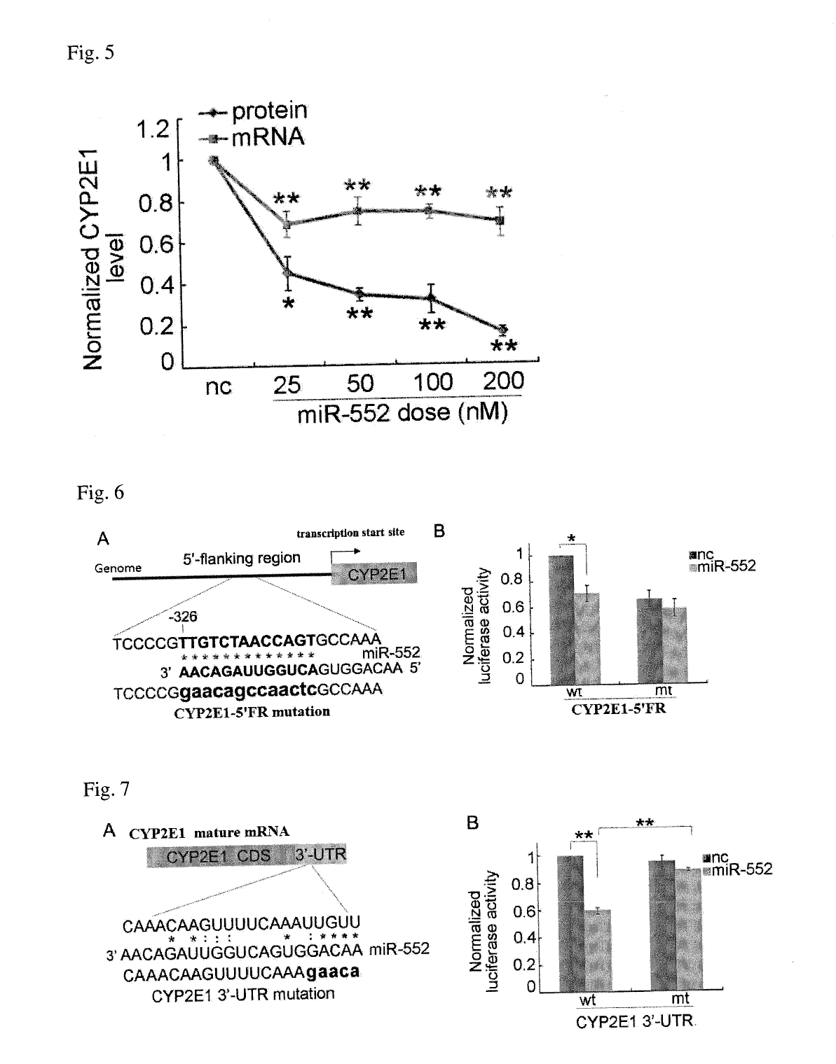

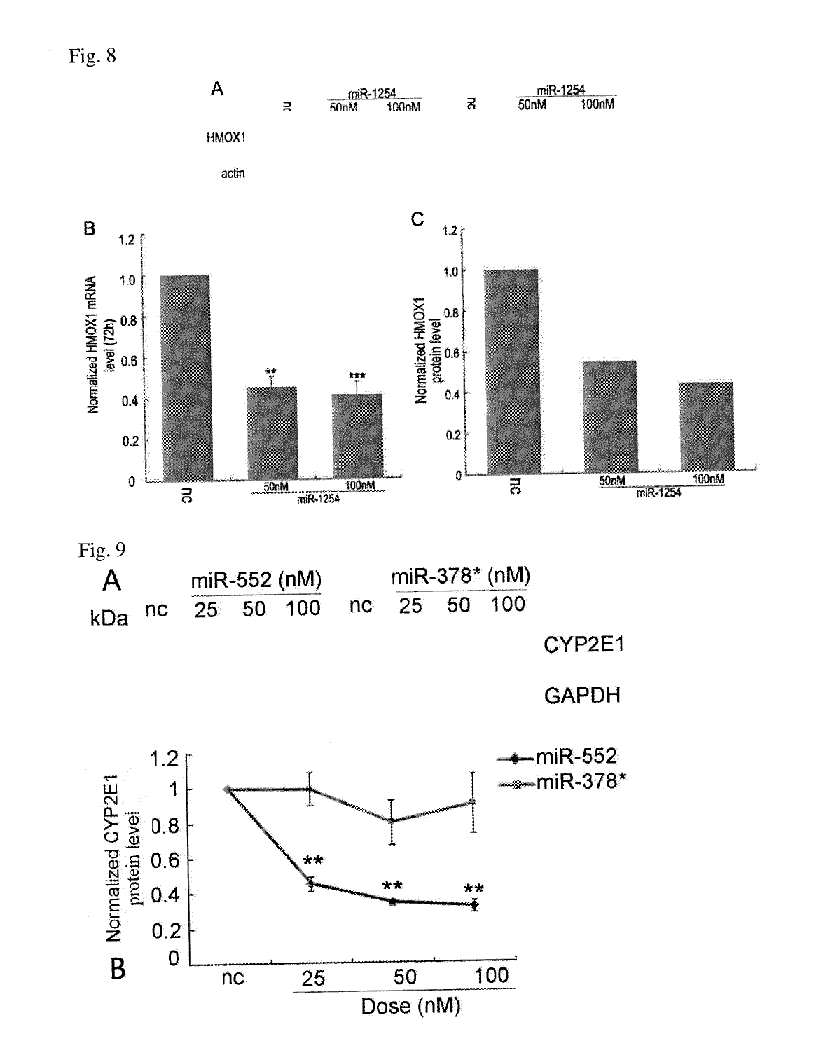

B. The statistical results corresponding to FIG. 4A, wherein gray detection was performed on the bands of CYP2E1 and GAPDH as internal reference by using ImageQuant Solutions software, and protein levels of CYP2E1 were normalized with nc as “1” according to the gray value of GAPDH so as to obtain the normalized protein expression level of CYP2E1.

FIG. 5 shows the inhibition of miR-552 on the mRNA expression level of CYP2E1. The protein expression level of CYP2E1 was down-regulated by miR-552 mimics in a dose-dependent manner, and the down-regulation of the mRNA expression level of CYP2E1 does not have such feature. The mRNA expression levels of CYP2E1 gene and GAPDH gene as internal reference were detected by using a Rotor Gene real-time fluorescence quantitative PCR instrument with Takara fluorescence quantitative PCR reagents. The mRNA expression levels of CYP2E1 were normalized with nc as “1” according to the expression level of GAPDH so as to obtain the normalized mRNA expression level of CYP2E1. * represents P<0.05 vs nc, ** represents P<0.01 vs nc.

FIG. 6 shows that the effects of the mutation in the miR-552 binding site of 5′-flanking region of CYP2E1 on the inhibitory ability of miR-552 at transcriptional level, wherein

A. The nucleotides at 9-21 of miR-552 can complementarily pair with the nucleotide sequence at 313-326 upstream from the transcription start site of CYP2E1, wherein the lowercase letters represent the mutated nucleotide sequence of the miR-552 binding site of the promoter region of CYP2E1.

B. Luciferase reporter assay demonstrated that miR-552 inhibited the activity of the promoter of the wide-type CYP2E1 and did not affect the activity of the promoter of CYP2Elwith mutation into the possible miR-552 binding site. The activities of renilla luciferase was normalized with nc as “1” according to the luciferase activity of firefly luciferase as internal reference so as to obtain the normalized luciferase activity. * represents P<0.05 vs nc.

FIG. 7 shows the effects of the mutation in miR-552 binding site of 3′-untranslated region of CYP2E1 on the inhibitory ability of miR-552 at post-transcriptional level, wherein

A. the mRNA 3′-UTR of CYP2E1 partially paired with miR-552 seed region, wherein the lowercase letters represent the mutated nucleotide sequence with mutations into the possible miR-552 binding site of mRNA 3′-UTR of CYP2E1.

B. Luciferase reporter gene assay demonstrated that miR-552 inhibited the activity of 3′-UTR of CYP2E1 and did not inhibit that of 3′-UTR with mutation into the possible miR-552 binding site. The activities of luciferase was normalized with nc as 1 according to the luciferase activity of renilla luciferase as internal reference so as to obtain the normalized luciferase activity. ** represents P<0.01 vs nc.

FIG. 8 shows the inhibition of miR-1254 on the mRNA and protein levels of HMOX1, wherein:

A. Protein immunoblot of evaluating the expression of HMOX1 after miR-1254 mimics was transfected into 293 cells, wherein beta-actin was used as the control of loading amount, and nc is negative control.

B. The protein and mRNA expression level of HMOX1 was down-regulated by miR-1254 mimics in a dose-dependent manner. The mRNA expression levels of HMOX1 and GAPDH as internal reference gene were detected by using Rotor Gene real-time fluorescence quantitative PCR instrument with Takara fluorescence quantitative PCR reagents. The mRNA expression levels of HMOX1 were normalized with nc as “1” according to the expression level of GAPDH so as to obtain the normalized mRNA expression level of HMOX1. ** represents P<0.01 vs nc, and *** represents P<0.001 vs nc.

C. The statistical results corresponding to FIG. 8A, wherein gray detection was performed on the bands of HMOX1 and beta-actin as internal reference by using ImageQuant Solutions software, and protein expression levels of HMOX1 were normalized with nc as “1” according to the gray value of actin so as to obtain the normalized protein expression level of HMOX1.

FIG. 9 shows that the obtained miR-552 has better silencing effect to CYP2E1 expression than the previously reported miR-378, wherein:

A. Protein immunoblot of evaluating the expression of CYP2E1 after transfecting miR-552 and miR-378 mimics into PLC/PRF/5 cells, wherein GAPDH was used as the loading control, the numbers in the figure refer to the name of microRNAs, and nc is negative control.

B. The statistical results corresponding to FIG. 9A, wherein gray detection was performed on the bands of CYP2E1 and GAPDH as internal reference gene by using ImageQuant Solutions software, and protein expression levels of CYP2E1 were normalized with nc as “1” according to the gray value of GAPDH so as to obtain the normalized protein expression level of CYP2E1. ** represents P<0.01 vs nc.

### BEST MODE

Source of the reagents and instruments in examples:

Human hepatoma cells PLC/PRF/5 and 293 cells were purchased from Shanghai Institutes for Biological Sciences, Chinese academy of Science;

The plasmids PGL4.10, PGL4.73 and psiCHECK2 were from Promega;

Luciferase activity assay Kit was from Promega;

Transfection reagents lipofectamine 2000 was from Invitrogen;

Cell culture medium was from Invitrogen;

Instrument for protein immunoblot was from BioRad;

Site-directed DNA mutagenesis kit was from Merck;

Instrument for real-time quantitative RT-PCR was from QIAGEN;

Reagents for real-time quantitative RT-PCR were from Haojia, Shanghai;

Antibodies for protein immunoblot of CYP2E1 were from Millipore;

Antibodies for protein immunoblot of HMOX1 were from Santa Cruz.

Unless otherwise specified, the methods used in this invention are conventional in the art.

### PREPARATION EXAMPLES

### Preparation Example 1

### Identification of microRNA 552 (miR-552)

The microRNA 552 (miR-552) with dual inhibitory ability was obtained from the following steps by using Cytochrome P450 2E1 (CYP2E1) as the target gene:

Step 1): microRNA binding site prediction was performed on the 5′-flanking region of 1 kb upstream from the transcription start site of CYP2E1, on which the promoter may be probably located, by using miRbase database to identify a series of microRNAs that may potentially bind to the promoter region of the target gene (FIG. 2);

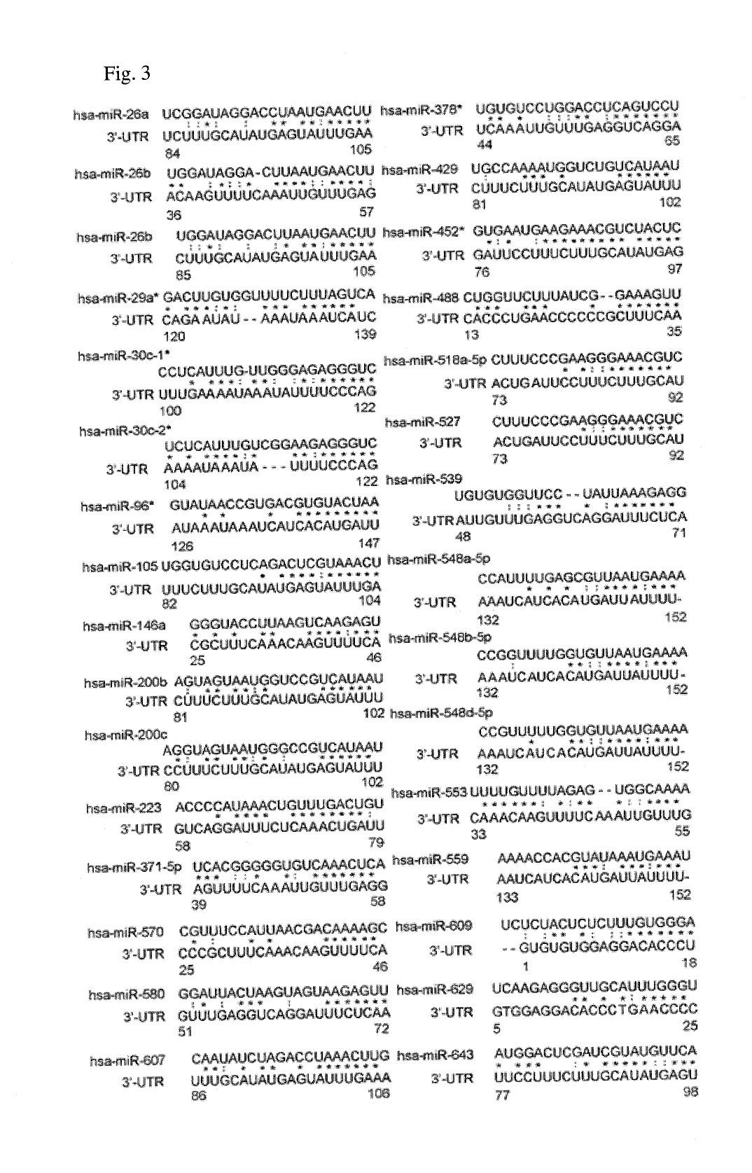

Step 2): microRNA binding site prediction was performed on the 3′-untranslated region of CYP2E1 by using Targetscan database to identify a series of microRNAs that may potentially bind to the 3′-untranslated region of CYP2E1 (FIG. 3);

Step 3): Chemically synthesized microRNA mimics were employed to evaluate the effects of the microRNAs from Steps 1) and 2) on the protein expression level of the target gene to obtain miR-552 that can significantly down-regulate the protein expression level of the target gene (FIG. 4).

Step 4): Chemically synthesized miR-552 mimics were employed to evaluate the effects of miR-552 on the mRNA expression level of CYP2E1. It was found that miR-552 can down-regulate mRNA expression level of CYP2E1 (FIG. 5).

Step 5): The miR-552 binding sites in the 5′-flanking region and the 3′-untranslated region of CYP2E1 were identified by using miRbase and Targetscan database prediction in combination with artificial alignment (FIGS. 2 and 7A). The miR-552 binding sites in the 5′-flanking region and the 3′-untranslated region of CYP2E1 were mutated by using site-directed mutagenesis (FIGS. 6 and 7A). The position and sequence of the wild-type 5′-flanking region and 3′-untranslated region were as follows:

the wild-type 5′-flanking region is positioned at 1352354624-1352355623, chromosome 10, 1000 by upstream from the transcription start site of CYP2E1 gene, human genome; and

the wild-type 3′-untranslated region is positioned at 1755-1912, the terminal of mature mRNA of CYP2E1 gene, and amounts to 158 bp.

The wild-type or mutated 5′-flanking region and 3′-untranslated region were inserted into luciferase reporter genes, respectively, wherein the 5′-flanking region was inserted into the multiple clone site of plasmid PGL4.10 and the 3′-untranslated region was inserted into the multiple clone site of plasmid psiCHECK2. The experiment demonstrated that (1) miR-552 can down-regulate the mRNA expression level of the target gene by binding to the specific site of the 5′-flanking region, suggesting that miR-552 has inhibitory ability at transcriptional level (FIG. 6B); (2) miR-552 can down-regulate the protein expression level of the target gene by binding to the specific site of the 3′-untranslated region, suggesting that miR-552 has inhibitory ability at translational level (FIG. 7B).

Step 6): miR-552 capable of significantly down-regulating both mRNA and protein expression levels of CYP2E1 with dual inhibitory ability at both transcriptional and post-transcriptional levels was identified by combining the data from steps 3), 4), and 5).

### Preparation Example 2

### Identification of microRNA 1254 (miR-1254)

Following the above-mentioned method, microRNA 1254 (miR-1254) with dual gene silencing ability at both transcriptional and post-transcriptional levels was identified by the same computer prediction and experimental method by selecting Hameoxygenase 1 (HMOX1) as the target gene (FIG. 8). This date confirmed that the present method is effective.

### EXPERIMENTAL EXAMPLES

### Example 1

Evaluate the inhibition of miR-552 on mRNA and protein expression levels of CYP2E1 in Human hepatoma PLC/PRF/5 cells.

MiR-552 mimics (25, 50, 100, 200 nM) were transfected into Human hepatoma PLC/PRF/5 cells, respectively. The variation in the protein expression level of CYP2E1 in cells was detected by protein immunoblot, and the variation in the mRNA expression level of CYP2E1 was detected by reverse transcription-real time quantitative PCR. As shown from FIGS. 4 and 5, MiR-552 decreased protein expression level of CYP2E1 in dose-dependent mode (FIG. 5), and slightly decreased mRNA expression level of CYP2E1 without dose dependency (FIG. 5).

### Example 2

### miR-552 Binds to the Promoter Region of CYP2E1 Gene Through its Non-Seed Region to Inhibit the Transcription Activity of CYP2E1

The effects of the cotransfection of miR-552 or the mutants thereof on the luciferase activity were evaluated by cloning the wild-type(wt) and miR-552 binding site mutants(mt) in the promoter region (5′-flanking region) of human CYP2E1 gene into PGL3 luciferase reporter system. As shown from FIG. 6, miR-552 can significantly inhibit the activity of the CYP2E1 promoter without suppressing the activity of the mutated promoter (FIG. 6B).

### Example 3

### MiR-552 Binds to the mRNA 3′UTR of CYP2E1 Through its Seed Region and Post-Transcriptionally Inhibit the Expression Level of CYP2E1

The effects of the cotransfection of miR-552 on the luciferase activity were evaluated by cloning the wild-type (wt) and miR-552 binding site mutants (mt) in the 3′UTR of human CYP2E1 into dual-luciferase reporter gene system. As shown from FIG. 7, miR-552 can significantly inhibit the luciferase activity of the 3′UTR of CYP2E1, and did not significantly suppress the luciferase activity of mutated 3′UTR.

### Example 4

### Evaluation on the Inhibition of miR-1254 on the mRNA and Protein Expression Levels of HMOX1 in Human Renal 293 Cells

MiR-1254 mimics (50, 100 nM) were transfected into human renal 293 cells, respectively. The variation in protein expression level of HMOX1 in cells was detected by protein immunoblot, and the variation in mRNA expression level of HMOX1 was detected by reverse transcription-real time quantitative PCR. As shown from FIG. 8, MiR-1254 can significantly decrease the protein expression level of HMOX1 in a dose-dependent manner (FIG. 8A, C) and the mRNA expression level of HMOX1 (FIG. 8B).

1. A method for screening a microRNA with gene silencing function at both transcriptional and post-transcriptional levels, comprising:
Step 1): predicting the microRNA binding site on the 5′-flanking region of 1 to 2 kb upstream from the transcription start site of target gene by using miRbase database, to identify the microRNA capable of binding to the promoter region of the target gene;
Step 2): predicting the microRNA binding site on the 3′-untranslated region of the target gene by using Targetscan database to identify the microRNA capable of binding to the 3′-untranslated region of the target gene;
Step 3): chemically synthesizing the microRNAs identified in Steps 1) and 2), evaluating their effects on the protein expression of the target gene, and thereby screening out the microRNA capable of down-regulating the protein expression of the target gene;
Step 4): evaluating the effect of the microRNA capable of down-regulating the protein expression of the target gene and obtained from Step 3) on the mRNA expression of the target gene, and thereby screening out the microRNA capable of down-regulating both mRNA and protein levels of the target gene;
Step 5): evaluating the microRNA for its ability to silence the target gene at both transcriptional and post-transcriptional levels by using site-directed mutagenesis and luciferase reporter gene methods, and thereby screening out the microRNA with gene silencing function at both transcriptional and post-transcriptional levels.

2. The method of claim 1, wherein in the Step 3), the method for evaluating the effects of the microRNA on the protein expression level of the target gene includes liposome transfection and protein immunoblot.

3. The method of claim 1, wherein in the Step 4), the method for evaluating the effects of the microRNA on the mRNA expression level of the target gene includes total RNA isolation, cDNA reverse transcription and real-time fluorescence quantitative PCR.

4. The method of claim 1, wherein the Step 5) further comprising:
(a) identifying the binding site of the 5′-flanking region of the target gene with the screened microRNA by using miRbase database prediction in combination with artificial alignment, mutating the binding site in the 5′-flanking region by using site-directed mutagenesis, inserting wild-type or mutated 5′-flanking regions into the renilla luciferase-carrying vector respectively, with the firefly luciferase-carrying vector used as an internal reference, transforming the two types of vectors into cells respectively, introducing the microRNA obtained from Step 4), evaluating the effect of the mutation of the microRNA binding site in the 5′ flanking region on the renilla luciferase activity, confirming the binding site of the 5′ flanking region of the target gene with the microRNA, and finally determining whether the microRNA has the regulatory ability at transcriptional level, and thereby screening out microRNA that has the regulatory ability at transcriptional level;
(b) identifying the binding site of the 3′UTR of the target gene with the screened microRNA using Targetscan database prediction in combination with artificial alignment, mutating the binding site in the 3′UTR via site-directed mutagenesis, inserting the wild-type or mutated 3′UTR into the firefly luciferase-carrying vector respectively, with the renilla luciferase self-carried by the vector used as an internal reference, transfecting the vectors carrying the wild-type or mutated 3′UTR into cells with the microRNA mimics, evaluating the effect of the mutation of the 3′UTR of the target gene on miRNA-induced inhibition on the firefly luciferase activity, confirming the binding site of the microRNA having the regulatory ability at transcriptional level obtained from step (a) to the 3′UTR of the target gene, and finally determining whether the microRNA has regulatory ability at post-transcriptional level, and thereby screening out the microRNA with gene silencing function at both transcriptional and post-transcriptional levels.

5. The method of claim 4, wherein the cell is PLC/PRF/5 liver cell line, 293 cell line, or 786-0 renal cell line.

6. The method of claim 1, wherein the target gene is cytochrome P450 2E1 gene or haemoxygenase 1 gene.

7. A microRNA with gene silencing function at both transcriptional and post-transcriptional levels obtained by the method of claim 1.

8. A method for silencing a gene in a subject, comprising:
administering to the subject an effective amount of the microRNA of claim 7.

9. A method of gene silencing, comprising performing gene silencing by using the microRNA obtained by the method of claim 1.
